(19) United States
(12) Patent Application Publication
(10) Pub. No.: US 2011/0040078 A1
(43) Pub. Date: Feb. 17, 2011

(54) PROCESS FOR THE PRODUCTION OF TELITHROMYCIN

(75) Inventors: Ingolf Macher, Woergl (AT); Dominic de Souza, Regau (AT)

Correspondence Address: Manelii Denison & Selter PLLC, 2000 M Street, 7th Floor, Washington DC, DC, 20036, US

(73) Assignees: Siegfried Wolf, Brixlegg (AT); SANDOZ AG, Basel (SU)

(21) Appl. No.: 12/739,194

(22) PCT Filed: Oct. 10, 2008
(86) PCT No.: PCT/EP2008/063655

§ 371 Date: Oct. 29, 2010

(30) Foreign Application Priority Data
Oct. 25, 2007 (EP) 07119257.9

(51) Int. Cl. C07H 17/08; C07D 401/04

(52) U.S. Cl. 536/7.1; 546/274.1

(57) ABSTRACT

The present invention relates to a process for the preparation of erythromysin derivatives, in particular telithromycin of formula (I) and its pharmaceutically acceptable salts, providing the isolated intermediates in crystalline form of superior stability and purity.

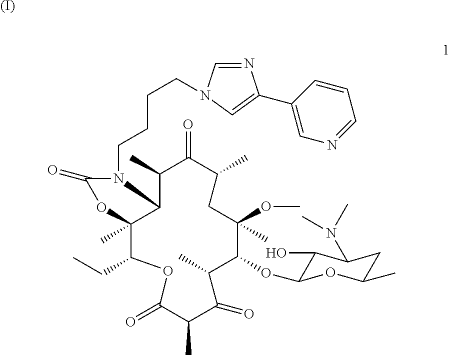

The present invention relates to a process for the preparation of compounds of formula (I) or its pharmaceutically acceptable salts.

Macrolides are a well known class of antibiotics. A novel class of macrolides with a common C-3 ketone group, called ketolides, has been discovered some years ago. Typically, they are derivatives of erythromycin, a well known and widely prescribed antibiotic for the treatment of respiratory tract infections.

EP 680967 describes such erythromycin derivatives and their non-toxic, pharmaceutically acceptable acid addition salts. Among them, also the compound 3-de[(2,6-dideoxy-3-C-methyl-3-O-methyl-α-L-ribohexopyranosyl)oxy]-11,12-dideoxy-6-O-methyl-3-oxo-12,11-[oxycarbonyl[[4-[4-(3-pyridinyl)-1H-imidazol-1-yl]butyl]imino]]erythromycin of the formula 1 with the international nonproprietary name of telithromycin is described.

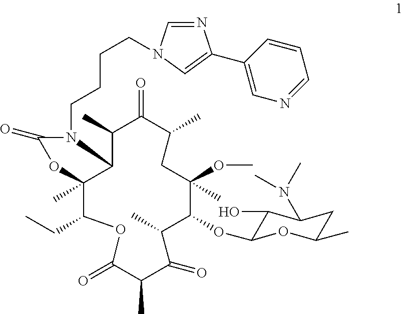

The process of EP 680967 which is also described in Curr. Med. Chem. 2001, 8, 1727-1758 (see Scheme 1) suffers several drawbacks in that it is complicated especially due to the high number of isolated intermediates. Moreover, it is very difficult to remove unreacted reagents and impurities formed during the reaction. The process for the synthesis of intermediate 8 (see Scheme 1) is described in Agouridas et al. J. Med. Chem. 1998, 41, 4080-4100 as well as in EP 0487411 and EP 0596802 and includes three necessary tedious column chromatographies, one precipitation and one crystallization. Further, said precipitation and crystallization do not yield high purity intermediates as the resulting products are obtained as brown solids. Further, the crystallization is performed in diethyl ether, which suffers serious disadvantages especially on production scale due to its high volatility, its property to form a wide range of highly explosive air/vapor mixtures and its pronounced tendency to form explosive peroxides. It is therefore raising major safety issues especially on production scale and makes special treatment necessary. Another disadvantage of said procedure is the use of moisture sensitive and pyrophoric sodium hydride. The process for the synthesis of telithromycin from intermediate 8 is described in Bioorg. Med. Chem. Lett. 1999, 9, 3075-3080 as well as in EP 680967. The described condensation of intermediate 8 with side chain 10 followed by deprotection suffers several drawbacks such as

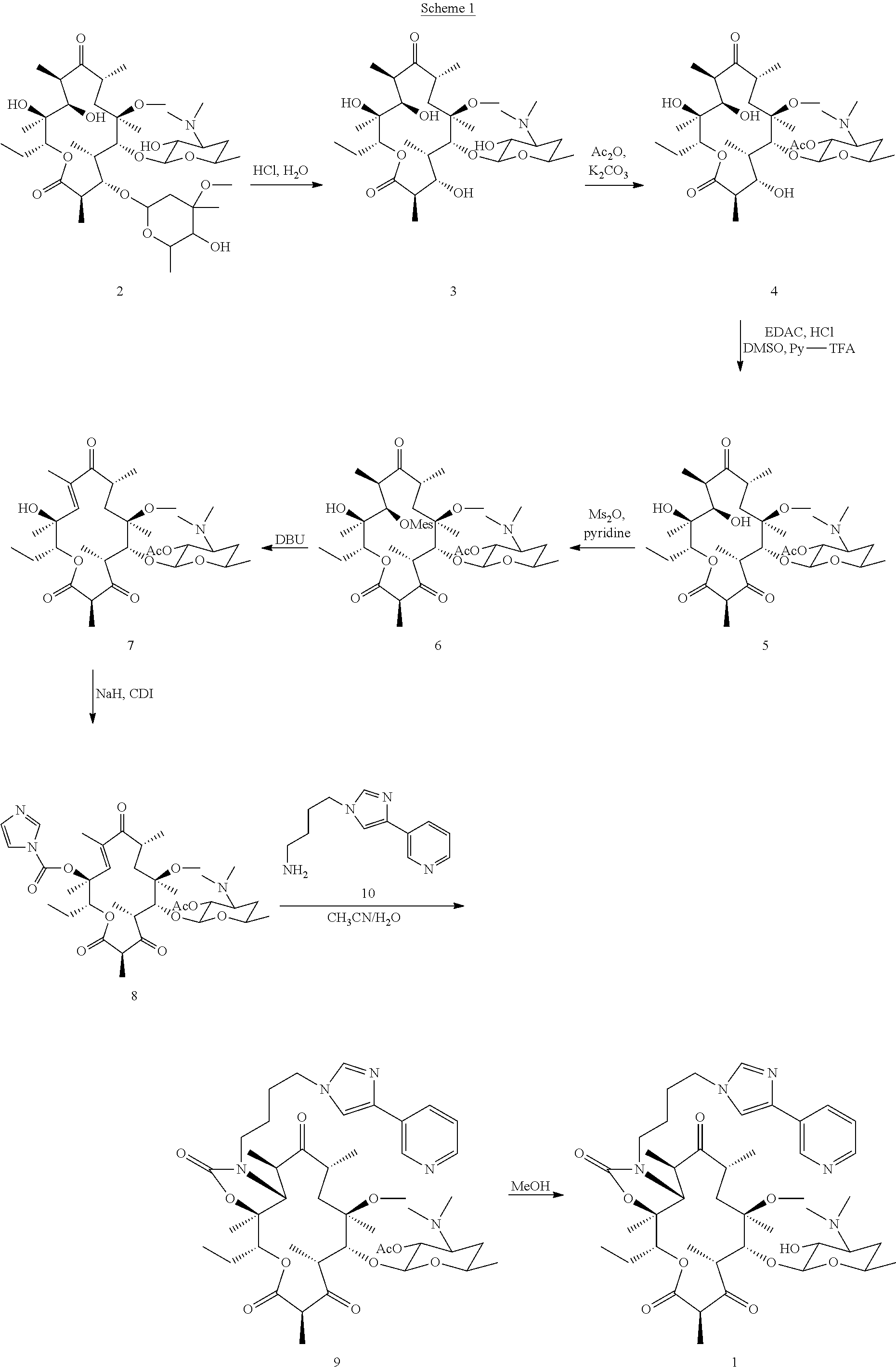

a) The side chain of formula 10 is produced by cleavage of the phthalimide in 4-(3-pyridyl)1H-imidazol-1-butanamide phthalimide of formula 17 by means of hydrazine hydrate, which is highly toxic and known to be a human carcinogen. It is further environmentally hazardous thus raising major safety issues especially on production scale and demanding special effluent treatment.
b) The handling of side chain 10 is difficult and burdensome especially at commercial production scale due to its physical properties being a highly viscous oil. Further, side chain 10 cannot be stored for longer time without considerable decomposition.
c) The condensation of intermediate 8 with side chain 10 is cumbersome and it is very difficult to remove impurities and side products formed during the reaction.
d) Therefore, the isolation and purification of the desired compound of the formula 1 needs a laborious column chromatography followed by a crystallization. Further, the crystallization is performed in diethyl ether, which suffers the serious disadvantages mentioned above.

A further process for the production of telithromycin is disclosed in WO 2005105821. This process is depicted in Scheme 2, wherein “R₁” and “R₂” are as defined in the referred patent, and overcomes some disadvantages of the process described above.

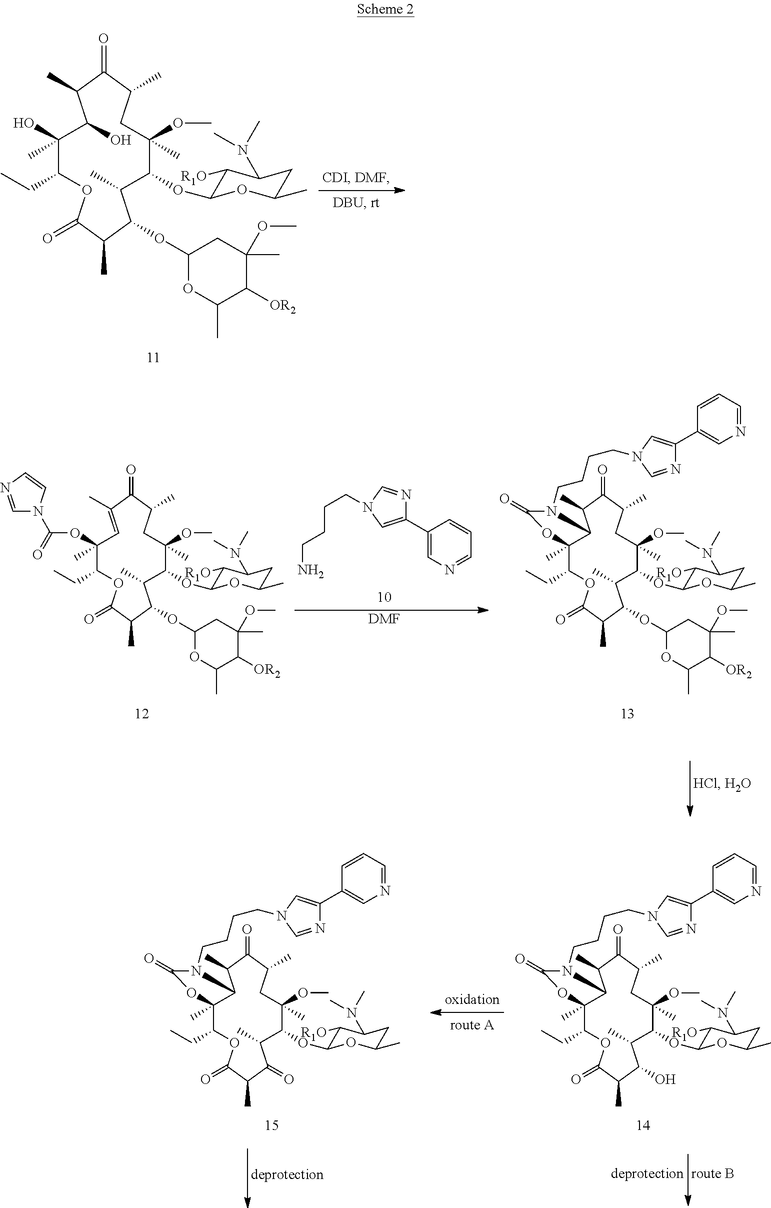

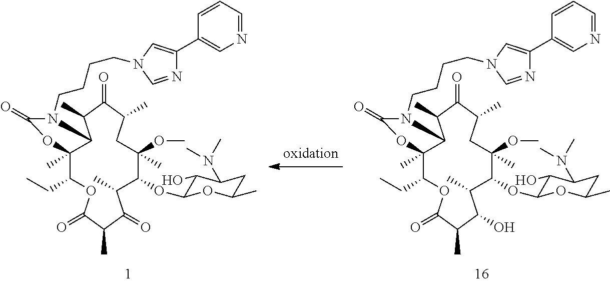

Reacting compound 11 with a large excess (5 eq.) of carbonyldiimidazole (CDI) in a polar, water miscible solvent (DMF, THF, CH₃CN) in the presence of a base (DBU, triethylamine, diisopropyl ethylamine) yields compound 12 which is isolated by precipitation. The resulting solid is dried and then reacted with compound 10 to give the condensation product 13 which is isolated via precipitation and dried. Next, the cladinose moiety is cleaved using standard conditions and the resulting product 14 is again isolated by precipitation and dried. The last two transformations, namely oxidation and deprotection, may be performed in either order. Oxidation of compound 14 (route A) using methods know to those skilled in the art gives compound 15 which was isolated simply by distilling off the organic solvent from the solution obtained after workup. Deprotection of compound 15 with an alcohol (optionally in the presence of a mineral acid) gives the desired compound 1 which is purified by recrystallization from diisopropyl ether or from methyl tert-butyl ether in combination with cyclohexanone. Alternatively following route B, deprotection of compound 14 using an alcohol (optionally in the presence of a mineral acid) yields compound 16 which is purified by crystallization from acetone. Oxidation of compound 16 gives the desired compound 1 which is purified by crystallization from diisopropyl ether.

Also this procedure suffers several drawbacks such as:
a) The referred process still has a high number of isolated intermediates that are dried prior to further transformation rendering the process tedious especially on large scale. Drying is a highly time consuming process requiring a lot of energy.
b) The side chain is introduced using the free amine 10 leading to the same problems concerning handling and storage as discussed above. Furthermore, the high cost side chain is introduced early in the reaction sequence leading to higher financial losses due to inevitable product losses in the remaining reaction steps.
c) Most intermediates are isolated via precipitation. It is very well known to those skilled in the art that precipitation is accompanied only with poor depletion of undesired compounds especially if compared to crystallization. This is especially true if the starting materials, side products and desired products have a similar molecular structure. Therefore, the purification needed to obtain telithromycin in a quality suitable for medicinal use is exclusively done at the end of the reaction sequence (route A) or on the final intermediate and the final desired product (route B).

There is thus a need for improved processes for the production of telithromycin and in particular for processes, which lend themselves to large scale production while providing highly pure telithromycin. This can be achieved by:
a) Using reagents that are stable, non toxic, non hazardous, do not raise safety issues and are easily handled in large quantities.
b) Isolating as few intermediates as necessary thus enhancing the industrial feasibility.
c) Purifying and isolating intermediates preferably by crystallization accompanied by good depletion of undesired compounds from the reaction mixture to ensure conversions which are nearly free of byproducts leading subsequently to highly pure telithromycin.
d) Handling not isolated intermediates in solution in order to guarantee practicability and improved throughput on industrial scale.

Another object of the invention is to provide a process for production of 4-[4-(3-pyridyl)imidazol-1-yl]butylamine (compound 10) avoiding toxic and environmentally hazardous hydrazine hydrate.

A further object of the invention is to provide a process for manufacturing telithromycin that involves the use of a stable, easy to handle derivative of compound 10.

Also, the object of the invention is the preparation of novel, solid, stable and easy to handle acid addition salts of compound 10 represented by the formula 18 and their use in the preparation of telithromycin.

Furthermore, the object of the invention is the preparation of compounds of formula 18 in crystalline form with high purity.

### SUMMARY OF THE INVENTION

The present invention relates to a new process for the production of telithromycin of formula 1 or its pharmaceutically acceptable salts characterized by the following steps:
a) Reacting commercially available 4-(3-pyridyl)1H-imidazol-1-butanamide phthalimide of formula 17 with an acid or a base to obtain 4-[4-(3-pyridyl)imidazol-1-yl]butylamine either directly in form of its acid addition salts represented by formula 18 in which “n” represents a number between 1-4 and “HA” represents an inorganic or organic acid or—if a base is employed—after acidic workup. Alternatively, 4-[4-(3-pyridyl)imidazol-1-yl]butylamine of formula 10 can be obtained as free amine via extraction from a basic aqueous mixture with an appropriate organic solvent.

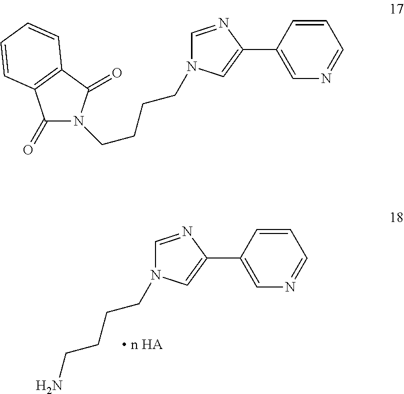

b) Crystallizing compounds of formula 19 which can be obtained without isolation of any intermediate from clarithromycin in high yield following the procedure described in WO 1997042205 from an appropriate solvent or a mixture of appropriate solvents to obtain compounds 19 where “R” is a hydroxyl protecting group in crystalline form with high purity.

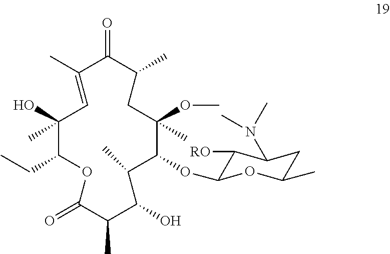

c) Oxidizing the resulting compounds 19 in the presence of an oxidizing agent to give compounds of formula 20 followed by crystallization from an appropriate solvent or a mixture of appropriate solvents to obtain compounds 20 in crystalline form with high purity.

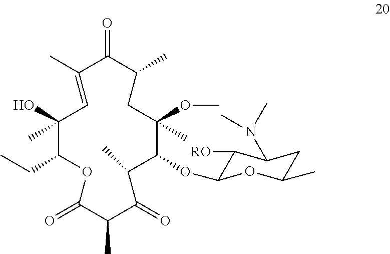

d) Reacting compounds of formula 20 with carbonyldiimidazole in the presence of a base to obtain compounds of formula 21.

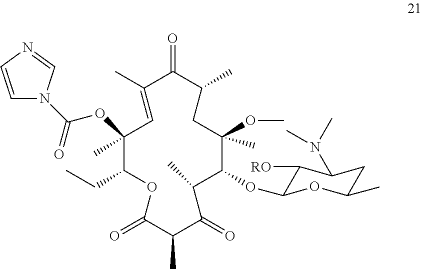

e) Condensing compounds of formula 21 with compounds of formula 18 in a suitable solvent in the presence of a base to give compounds of the formula 22.

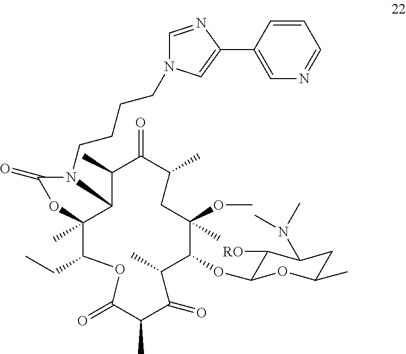

f) Removing the protecting group “R” in compounds of the formula 22 in the presence of an alcohol preferably in the presence of an aqueous base to give telithromycin of formula 1 as a single isomer.
g) Crystallizing thus obtained telithromycin from an appropriate solvent or a mixture of appropriate solvents to give telithromycin of formula 1 in high purity.

Furthermore, the present invention relates to novel compounds of the formula 18, where “n” represents a number between 1-4 and “HA” represents an inorganic or organic acid and their use in the preparation of telithromycin.

Further, the invention relates to a process for the preparation of telithromycin which is characterized by isolation of intermediates in their crystalline form with high purity.

Also, the invention relates to novel crystalline forms of compounds of the formula 18, 19a (R═Ac), 20a (R═Ac), 22a (R═Ac), and 24 characterized by their X-ray powder diffraction pattern (XRPD).

The invention describes a straightforward, ecological and economical process for the production of telithromycin of formula 1 which is distinguished by isolating only two intermediates (19 and 20) in their crystalline form.

Another aspect of the invention is that intermediates which are not isolated (23, 24, 21, and 22) are further processed in solution. It may be beneficial to isolate intermediate 23 as a precipitate. In this case intermediate 23 is further processed without drying.

### BRIEF DESCRIPTION OF THE FIGURES

FIG. 1: XRD spectrum of compound 23.

FIG. 2: XRD spectrum of compound 24.

FIG. 3: XRD spectrum of compound 19a.

FIG. 4: XRD spectrum of compound 20a.

FIG. 5: XRD spectrum of compound 1.

FIG. 6: XRD spectrum of 4-[4-(3-pyridyl)imidazol-1-yl]butylamine*3HCl*2H₂O.

FIG. 7: XRD spectrum of compound 22a.

### DETAILED DESCRIPTION OF THE INVENTION

The new process for the production of 4-[4-(3-pyridyl)imidazol-1-yl]butylamine of formula 10 and its novel acid addition salts of formula 18 is outlined in Scheme 3, wherein “HA” represents an organic or inorganic acid or mixtures thereof and “B” represents an organic or inorganic base or mixtures thereof.

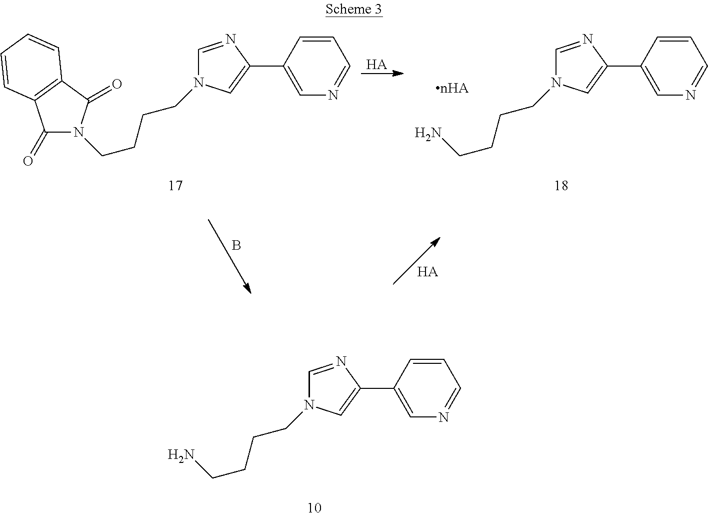

The substitution of toxic and environmentally hazardous hydrazine hydrate can be accomplished by stirring commercially available compound 17 in at least one aqueous acid (HA) or at least one base (B). The acid HA used in this step is preferably hydrochloric acid. If an acid is used in this step the acid addition salts of formula 18 can be obtained by removal of phthalic acid and addition of the resulting aqueous solution to an anti solvent such as an alcohol, preferably the alcohol is 2-propanol. The base in this step is preferably sodium hydroxide or potassium hydroxide. If a base is used in this step for the cleavage of the phthalimide the acid addition salts of formula 18 can be obtained by extractive workup with an acid HA as defined above and addition of the resulting aqueous solution to an anti solvent as defined above.

An embodiment of the invention is that the acid addition salts of formula 18 can be obtained in analytically pure form via crystallization. This is especially useful as it is very difficult to purify the neutral compound 10 otherwise.

Unlike the free amine of formula 10 the acid addition salts of formula 18 thus obtained can be stored for several months without any decomposition.

The new process for the production of telithromycin of formula 1 is outlined in Scheme 4 where “R” represents an hydroxyl protecting group.

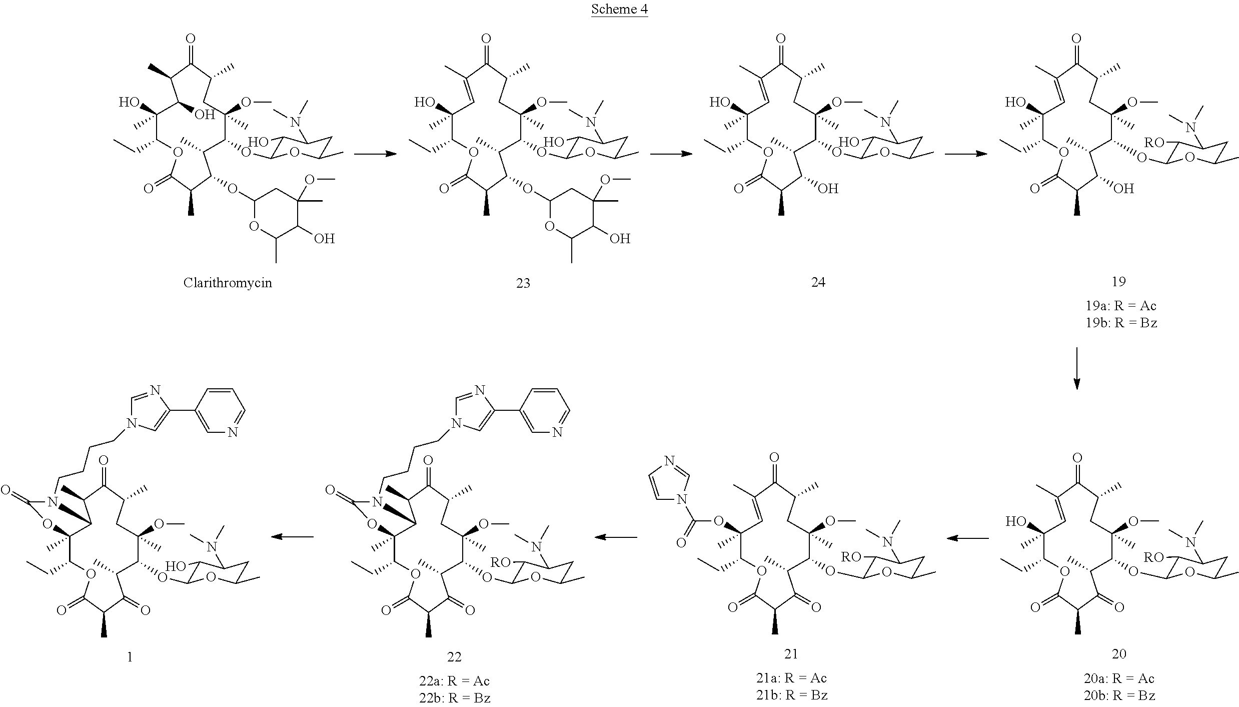

Clarithromycin (6-O-methylerythromycin; CAS reg. no: 81103-11-9) can be transformed to (10E)-10,11-didehydro-11-deoxy-6-O-methylerythromycin (CAS reg. no: 144604-03-5) of formula 23 according to methods described for example in WO 1997042205; WO 2004108745; Baker et al. J. Org. Chem. 1988, 53, 2340-2345; Elliott et al. J. Med. Chem. 1988, 41, 1651-1659; Ma et al. J. Med. Chem. 2001, 44, 4137-4156; U.S. Pat. No. 6,075,011;WO 2003072588, and EP 559896. Compound 23 can be further processed without isolation to compound 24 according to Elliott et al. J. Med. Chem. 1988, 41, 1651-1659. Alternatively, compound 23 may be precipitated from the reaction mixture by addition of an anti solvent (e.g. water) collected by filtration and washed according to EP 559896. If compound 23 is collected by precipitation, filtration, and washing with water it can be further processed without drying.

The cladinose moiety of compound 23 can be cleaved to give (10E)-3-O-de(2,6-dideoxy-3-C-methyl-3-O-methyl-α-L-ribo-hexopyranosyl)-10,11-didehydro-11-deoxy-6-O-methylerythromycin (CAS reg. no: 198782-59-1) of formula 24 according to methods described for example in Elliott et al. J. Med. Chem. 1988, 41, 1651-1659; WO 1997042205, and U.S. Pat. No. 6,720,308 by treatment with aqueous hydrochloric acid. After basification the resulting suspension is extracted with an appropriate organic solvent to give a solution of compound 24. It is preferred to extract with the aprotic solvent used in the next step and process compound 24 further to compounds of formula 19 in solution. Most preferably, methylene chloride is used for the extraction. Alternatively, compound 24 can be obtained as precipitate after basification and filtration.

An embodiment of the invention is that compound 24 can be obtained in analytically pure form by crystallization from an appropriate solvent or a mixture of appropriate solvents. If necessary an anti-solvent may be added. Preferred solvents for the crystallization of compound 24 are polar protic and polar aprotic solvents such as alcohols, esters and ketones. Preferred anti-solvents are among others water, hydrocarbons and ethers.

Selective protection of the 2′-hydroxy function of compound 24 using at least one suitable hydroxy group protecting agent in at least one aprotic solvent gives compounds of formula 19. If compound 24 is obtained in solution as described above it is preferred to use this solution.

Suitable hydroxy group protecting agents are listed for example in T. W. Greene, “Protective Groups in Organic Chemistry”, John Wiley & Sons, New York (1981). Preferably, the hydroxy group is protected as an ester using acetyl chloride, acetic anhydride, benzoyl chloride or benzoic anhydride as protecting agents in the presence of an appropriate base. Most preferably acetic anhydride is used to give (10E)-2′-O-acetyl-3-O-de(2,6-dideoxy-3-C-methyl-3-O-methyl-α-L-ribo-hexopyranosyl)-10,11-didehydro-11-deoxy-6-O-methylerythromycin (CAS reg. no: 198782-60-4) of formula 19a. In order to obtain telithromycin of formula 1 in high purity it is preferred to purify compounds of the formula 19 by crystallization, especially if the synthetic strategy does not favor the isolation of compounds 23 and 24. An embodiment of the invention is that compounds of the formula 19 can be obtained in analytically pure form by crystallization from an appropriate solvent or a mixture of appropriate solvents. If necessary an anti-solvent may be added. Preferred solvents for the crystallization of compound 19 are polar protic and polar aprotic solvents such as alcohols, esters and ketones. Preferred anti-solvents are among others water, hydrocarbons and ethers.

Oxidation of compounds of the formula 19 to give compounds of the formula 20 are carried out by employing commonly used oxidizing reagents such as activated dimethyl sulfoxide (DMSO) and related reagents (e.g. dimethylsulfide activated with N-chlorosuccinimide) as described in Tidwell Synthesis 1990, 857-870 and modifications thereof. The oxidation can also be carried out using Dess-Martin reagent, manganese-, chromium- or selenium reagents, tertiary amine oxides or by any above oxidant in the presence of at least one phase transfer catalyst. Thus oxidation of compound 19a gives (10E)-2′-O-Acetyl-3-de[(2,6-dideoxy-3-C-methyl-3-O-methyl-α-L-ribo-hexopyranosyl)oxy]-10,11-didehydro-11-deoxy-6-O-methyl-3-oxo-erythromycin (CAS reg. no: 214694-76-5) of formula 20a. To improve the quality of compounds of formula 20, they may be crystallized. An embodiment of the invention is that compounds of the formula 20 can be obtained in analytically pure form by crystallization from an appropriate solvent or a mixture of appropriate solvents. If necessary an anti-solvent may be added. Preferred solvents for the crystallization of compound 20 are polar protic and polar aprotic solvents such as alcohols, esters and ketones. Preferred anti-solvents are among others water, hydrocarbons and ethers.

The formation of C-12 imidazoyl carbamates which are essential intermediates for the preparation of telithromycin of formula 1 is usually carried out in N,N-dimethylformamide (DMF), tetrahydrofuran (THF), acetonitrile and mixtures thereof with a 3-5 fold molar excess of carbonyldiimidazole (CDI) in the presence of a 2-4 fold molar excess of an organic or inorganic base as described in EP 680967, EP 0487411, EP 0596802, and WO 2005105821. Further, in publications describing the preparation of such imidazolyl carbamates of erythromycin type macrolides which are structurally similar to compounds 8, 12, and 21 always the same solvents and similar excesses of CDI and base are used (see e.g. Elliott et al. J. Med. Chem. 1988, 41, 1651-1659; WO 2006129257; US 20060135447; Baker et al. J. Org. Chem. 1988, 53, 2340-2345).

From our process improvement activities we learned that the use of methylene chloride as solvent leads to a dramatic reduction of the necessary amount of CDI and base rendering the process commercially much more feasible. Thus, reacting compounds 20 in methylene chloride at temperatures between −20 and 20° C. with 1-1.5 molar equivalents of CDI and 1-1.5 molar equivalents of an appropriate organic or inorganic base leads to complete conversion of compounds 20 to compounds 21. It is preferred to use 1.25 molar equivalents of CDI and 1.25 molar equivalents of a base. Preferably, an organic base such as DBU, triethylamine, diisopropylethylamine, 1,1,3,3-tetramethylguanidine (TMG) is used. Most preferably the base is DBU. After aqueous extraction it is further preferred not to isolate compounds 21 but to transform them further in solution. If the next reaction is not performed in methylene chloride a solvent exchange to the desired solvent is easily performed.

The condensation of C-12 imidazoyl carbamates with primary amines to form cyclic carbamates is described for example in EP 680967, WO 2005105821, Elliott et al. J. Med. Chem. 1988, 41, 1651-1659; WO 2006129257; US 20060135447; Baker et al. J. Org. Chem. 1988, 53, 2340-2345. Generally, these reactions are carried out in acetonitrile, DMF or mixtures thereof optionally in the presence of water and an external base. Often the reaction is carried out at elevated temperatures (40-80° C.). All publications describe the use of free primary amines for the above mentioned condensation. For the preparation of telithromycin of formula 1 the amine 4-[4-(3-pyridyl)imidazol-1-yl]butylamine (compound 10) has to be used. The use of free amine of formula 10 is accompanied with a lot of disadvantages, especially on industrial scale. In form of the free amine compound 10 is difficult to handle on large scale being a brown, highly viscous oil. Further, as compound 10 is not stable in its free amine form it is necessary to prepare it just prior to use. Furthermore, as mentioned above it is difficult to purify compound 10 as a free amine. Therefore, compound 10 is generally used without purification as described in EP 680967 and WO 2005105821. In order to obtain telithromycin of formula 1 in high purity without tedious purification procedures it is desirable to use a pure amine for the condensation reaction. As described above, compound 10 can be purified by crystallization in the form of its acid addition salts of formula 18. Such addition salts are stable for storage and—being crystalline solids—easy to handle. It is an embodiment of the invention to directly use acid addition salts of formula 18 for the preparation of telithromycin of formula 1. Thus, reacting compounds of the formula 21 with compounds of the formula 18 in the presence of an appropriate base in a polar aprotic solvent such as methylene chloride, acetonitrile and DMF or mixtures thereof gives protected telithromycin of formula 22. Preferably, an organic base such as DBU, triethylamine, diisopropylethylamine, 1,1,3,3-tetramethylguanidine (TMG) is used. Further, it is preferred to run the reaction at temperatures of 20-80° C., most preferably at 20-40° C. The reaction of compound 21a under these conditions gives 2′-O-Acetyl-3-de[(2,6-dideoxy-3-O-methyl-3-O-methyl-α-L-ribohexopyranosyl)oxy]-11,12-dideoxy-6-O-methyl-3-oxo-12,11-[oxycarbonyl[[4-[4-(3-pyridinyl)-1H-imidazol-1-yl]butyl]imino]]erythromycin (CAS reg. no: 306770-59-2) of formula 22a.

An embodiment of the invention is that compounds of the formula 22 can be obtained in analytically pure form by crystallization from an appropriate solvent or a mixture of appropriate solvents. If necessary an anti-solvent may be added. Preferred solvents for the crystallization of compound 22 are polar protic and polar aprotic solvents such as alcohols, esters and ketones. Preferred anti-solvents are among others water, hydrocarbons and ethers. It is preferred not to isolate compounds 22 in solid form but continue the process in solution after aqueous workup.

Removal of the 2′-protecting group is carried out as described e.g. in T. W. Greene, “Protective Groups in Organic Chemistry”, John Wiley & Sons, New York (1981). In the case of ester protecting groups the cleavage is preferred to be carried out by alcoholysis. The alcohol preferred in this step is selected from a group comprising of methanol, ethanol, n-propanol, isopropyl alcohol, tert-butyl alcohol, n-butanol or mixtures thereof. The preferred alcohols are methanol and ethanol. This step can also be carried out in the presence of an aqueous base such as NaOH, KOH, etc. The reaction is carried out at a temperature of 0-100° C., preferably at 0-20° C. Thus obtained telithromycin of formula 1 can be crystallized after workup from an appropriate solvent. If necessary an anti-solvent may be added. Preferred solvents for the crystallization of compound 1 are polar protic and polar aprotic solvents such as alcohols, esters and ketones. Preferred anti-solvents are among others water, hydrocarbons and ethers.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention in any manner.

### Examples

### Example 1

### Preparation of (10E)-2′-O-Acetyl-3-O-de(2,6-dideoxy-3-C-methyl-3-O-methyl-α-L-ribo-hexopyranosyl)-10,11-didehydro-11-deoxy-6-O-methylerythromycin (compound 19a)

### Step 1: (10E)-10,11-Didehydro-11-deoxy-6-O-methylerythromycin (compound 23)

Clarithromycin (200 g) is suspended in a mixture of ethylene carbonate (200 g) and triethylamine (400 mL). The suspension is stirred vigorously under nitrogen and heated to reflux until completion of the reaction is determined by HPLC analysis. The mixture is cooled to 50° C. and water (150 mL) is added. The resulting precipitate is collected by filtration and washed with water. The product thus obtained can be employed in the next step without drying or further purification.

Drying under vacuum at 40° C. yields 152 g of (10E)-10,11-Didehydro-11-deoxy-6-O-methylerythromycin which can be recrystallized from ethanol/water=1/1 to give 135 g of analytically pure title compound.

¹³C-NMR (CDCl₃): 207.81, 175.59, 142.96, 139.14, 103.51, 96.86, 80.48, 80.32, 79.6, 78.9, 78.37, 73.68, 72.96, 71.34, 69.23, 66.03, 65.84, 51.11, 49.86, 45.52, 40.68, 40.68, 40.68, 37.68, 35.45, 29.11, 22.77, 22.5, 21.93, 21.82, 21.23, 18.98, 18.98, 16.02, 13.68, 11.03, 9.98 ppm.

mp: 256-258° C.

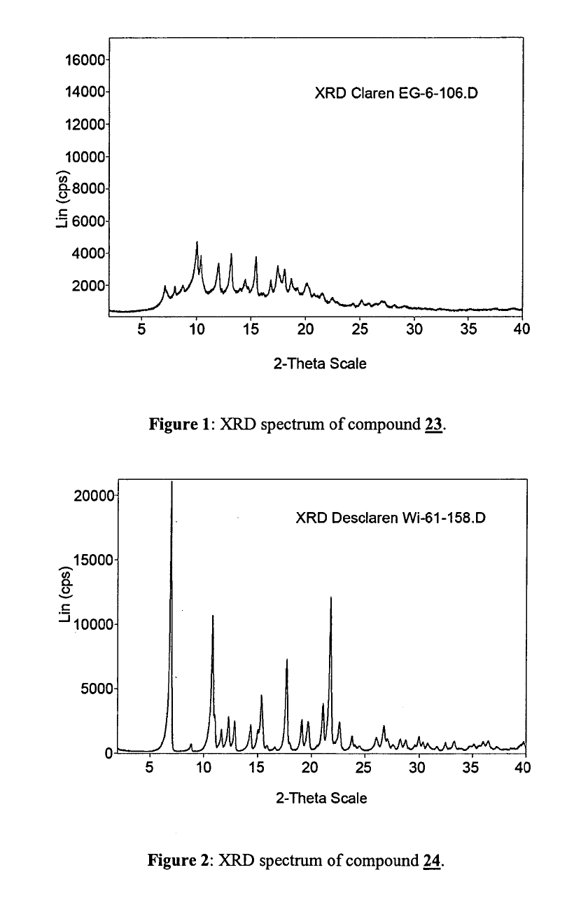

XRD spectrum of compound 23 (2-θ values): 8.1; 8.8; 10.0; 10.4; 12.0; 13.2; 14.5; 15.5; 16.9; 17.5; 18.1; 18.7; 20.2; 21.5; 22.5; 25.1; 27.1 (FIG. 1).

### Step 2: (10E)-3-O-De(2,6-dideoxy-3-C-methyl-3-O-methyl-α-L-ribo-hexopyranosyl)-10,11-didehydro-11-deoxy-6-O-methylerythromycin (compound 24)

The wet product from step 1 is suspended in 2.5 L of 0.5N aqueous HCl and stirred at ambient temperature until cleavage of cladinose is complete. Methylene chloride (750 mL) is added and the pH is adjusted to 11 by addition of 10N aqueous NaOH. The aqueous layer is extracted with methylene chloride (250 mL). The combined organic layers are washed with water (3×350mL) and then concentrated at atmospheric pressure to give a syrup which can be used in the next step without further purification.

Alternatively, the syrup can be crystallized from methanol/water=2/3 to give 101 g of analytically pure title compound.

¹³C-NMR (CDCl₃): 208.16, 177.13, 141.99, 139.02, 107.22, 92.39, 81.31, 79.62, 78.09, 73.88, 70.93, 70.12, 65.98, 48.63, 44.72, 40.67, 38.72, 37.34, 36.82, 28.68, 21.76, 21.23, 20.75, 20.52, 16.54, 16.16, 13.32, 10.91, 8.04 ppm.

mp: 114-116° C.

XRD spectrum of compound 24 (2-θ values): 7.0; 10.8; 11.6; 12.3; 12.9; 14.4; 15.4; 17.8: 19.1; 19.7; 21.1; 21.8; 22.6; 23.8; 26.0; 26.7; 28.3; 28.8; 30.0 (FIG. 2).

### Step 3: (10E)-2′-O-Acetyl-3-O-de(2,6-dideoxy-3-C-methyl-3-O-methyl-α-L-ribo-hexopyranosyl)-10,11-didehydro-11-deoxy-6-O-methylerythromycin (compound 19a)

The syrup obtained above is dissolved in methylene chloride (600 mL) and triethylamine (64 mL) and acetic anhydride (46.3 g) are added and the mixture is stirred at ambient temperature until completion of the reaction is determined by HPLC analysis. Methanol (22 mL) is added and after stiffing for 15 min the solution is extracted with 5% aqueous NaHCO₃ (500 mL) and water (500 mL). The organic layer is concentrated and the resulting residue is dissolved in isopropyl acetate (250 mL) at 50-60° C. The resulting solution is seeded and n-heptane (750 mL) is slowly added at 50° C. The crystal suspension is cooled to ambient temperature and stirred for 2 h. The crystalline product is isolated by filtration washed with isopropyl acetate/n-heptane=1/3 and dried under vacuum at 50° C. to give 104 g of the title compound in analytically pure form.

¹³C-NMR (CDCl₃): 208.15, 176.24, 170.60, 141.38, 139.85, 103.02, 88.28, 80.42, 79.78, 77.85, 73.71, 72.24, 69.61, 64.55, 49.24, 44.68, 41.14, 38.11, 37.83, 37.56, 30.55, 21.85, 21.7, 21.61, 21.06, 20.66, 17.37, 15.97, 13.91, 10.99, 8.35 ppm.

mp: 187-190° C.

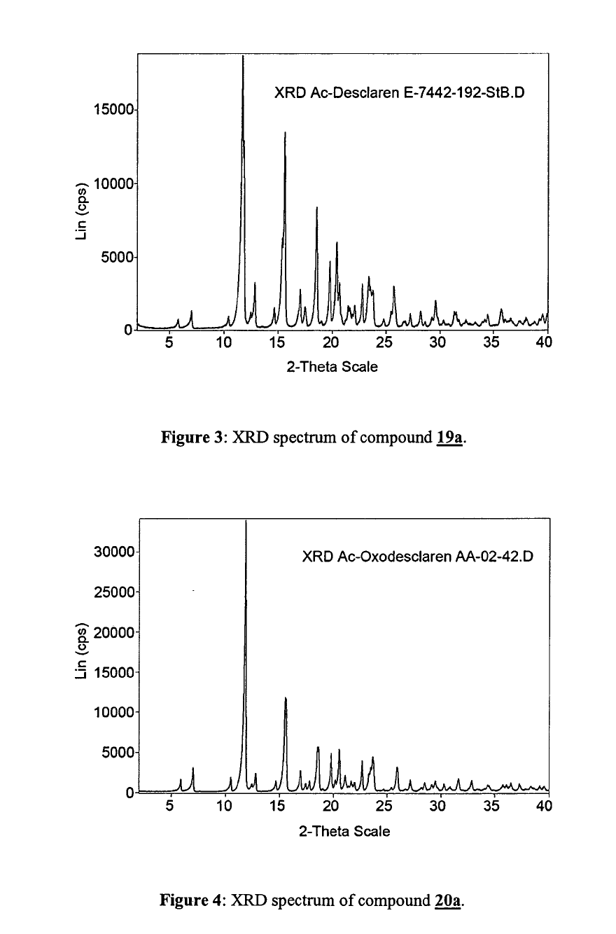

XRD spectrum of compound 19a (2-θ values): 5.8; 7.0; 10.4; 11.7; 12.9; 14.7; 15.6; 17.0; 17.5; 18.5; 19.8; 20.4; 20.7; 21.5; 22.1; 22.8; 23.4; 25.7; 27.3; 28.2; 29.6 (FIG. 3).

### Example 2

### Preparation of (10E)-2′-O-Acetyl-3-de[(2,6-dideoxy-3-C-methyl-3-O-methyl-α-L-ribo-hexopyranosyl)oxy]-10,11-didehydro-11-deoxy -6-O-methyl-3-oxo-erythromycin (compound 20a)

To a solution of compound 19a (60 g) in acetone (250 mL) is added diisopropylethylamine (128 g) and the mixture is cooled to −10° C. To this mixture a solution of pyridine*SO₃ (79 g), pyridine (39 g) and DMSO (232 g) is added during 20 min keeping the temperature at −10° C. After stiffing for 5 min complete conversion is detected by HPLC analysis. Water (1100 mL) and methylene chloride (570 mL) are added slowly and the resulting mixture is stirred for 20 min. The organic layer is washed with water and concentrated to give a syrup that is recrystallized from isopropyl alcohol at 70-20° C. The resulting suspension is cooled to −20° C. and the crystals are collected by filtration to give 49 g of the title compound in analytically pure form.

¹³C-NMR (CDCl₃): 207.48, 205.08, 170.17, 170.14, 142.55, 139.16, 102.22, 81.67, 81.41, 78.68, 77.46, 73.70, 71.95, 69.50, 63.94, 51.60, 50.79, 47.51, 41.06, 40.54, 38.84, 30.77, 22.79, 22.35, 21.78, 21.40, 19.30, 15.19, 14.42, 13.94, 11.33 ppm.

mp: 228-233° C.

XRD spectrum of compound 20a (2-θ values): 5.9; 7.0; 10.5; 11.8: 12.8; 14.7; 15.5; 16.9; 17.8; 18.5; 19.8; 20.5; 21.1; 22.7; 23.7; 25.9; 27.1; 29.5 (FIG. 4).

### Example 3

### Preparation of 3-De[(2,6-dideoxy-3-C-methyl-3-O-methyl-α-1-ribohexopyranosyl)oxy]-11,12-dideoxy-6-O-methyl-3-oxo-12,11-[oxycarbonyl[[4-[4-(3-pyridinyl)-1H-imidazol-1-yl]butyl]imino]]erythromycin (telithromycin, compound 1)

To a suspension of compound 20a (32 g) in methylene chloride (230 mL) is added DBU (11.8 g) and CDI (12.6 g) at −10° C. After completion of the reaction is detected by HPLC analysis the pH is adjusted to 6 with 10% aqueous acetic acid and the organic layer is washed with water (2×250 mL) and concentrated. Acetonitrile (320 mL) is added and the solution is again concentrated at 30° C. and 300 mbar. To the resulting solution is added 4-[4-(3-pyridyl)imidazol-1-yl]butylamine3HCl*2H₂O (34 g, prepared according to example 4) and TMG (40 g). The mixture is stirred at ambient temperature until completion of the reaction is detected by HPLC analysis. Water (340 mL) and methylene chloride (340 mL) are added and the pH is adjusted to 6 with 10% aqueous acetic acid. The organic layer is washed with water (150 mL) and concentrated. Ethanol (125 mL) is added and the resulting solution is cooled to 0° C. After addition of 10% aqueous NaOH (125 mL) the mixture is stirred at 0° C. until completion of the reaction is detected by HPLC analysis. The pH of the mixture is adjusted to 7 with 20% aqueous HCl and the organic solvents are evaporated. Ethyl acetate (600 mL) is added and the pH is adjusted to 8 with 20% aqueous NaOH. The organic layer is washed with water and concentrated to 130 g. The solution is heated to 40° C. , diisopropyl ether (200 mL) is added and seeded. After stirring for 1 h at 40° C. another portion of diisopropyl ether (200 mL) is added. The crystals are collected by filtration to give 27 g of the title compound in analytically pure form.

¹³C-NMR (CDCl₃): 216.71, 203.91, 170.28, 157.72, 148.02, 146.88, 139.53, 138.17, 132.33, 130.69, 123.86, 115.90, 104.40, 82.59, 80.13, 78.64, 77.92, 70.73, 70.04, 66.24, 60.63, 51.63, 50.24, 48.07, 47.25, 45.31, 42.95, 40.64, 39.96, 39.39, 29.01, 28.52, 24.68, 22.63, 21.59, 20.15, 18.80, 16.28, 15.07, 14.74, 14.30, 10.91 ppm.

mp: 185° C.

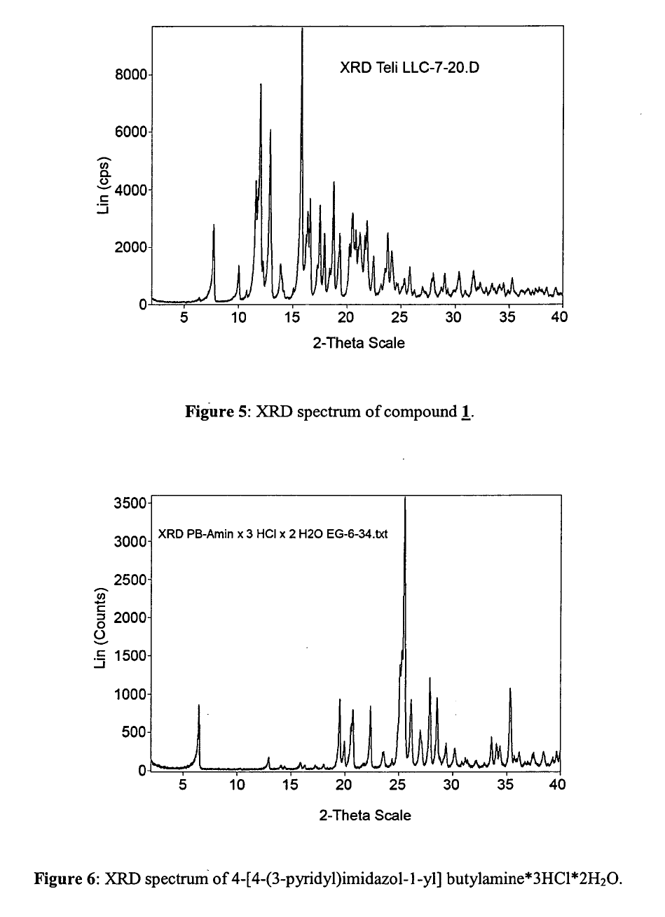

XRD spectrum of compound 1 (2-θ values): 7.7; 10.0; 11.6; 12.0; 12.9; 13.9; 15.8; 16.6; 17.5; 17.9; 18.8; 19.3; 20.5; 20.8; 21.2; 21.8; 22.4; 23.8; 24.1; 25.8; 28.8; 29.0 (FIG. 5).

### Example 4

### Preparation of 4-[4-(3-pyridyl)imidazol-1-yl]butylamine*3HCl*2H₂O

Version 1: Cleavage of Phthalimide with HCl

A solution of 4-(3-pyridyl)1H-imidazol-1-butanamide phthalimide (20 g) in 6N aqueous HCl (50 mL) is refluxed until completion of the reaction is detected by HPLC analysis. The mixture is cooled to 0° C. and stirred for 1 h. The precipitate is filtered off and washed with water (3×5mL). The filtrate and washings are combined and slowly added to 2-propanol (650mL) at 45° C. The resulting suspension is aged for 2 h at ambient temperature and for another hour at 0° C. The crystals are collected by filtration, washed with 2-propanol and dried at ambient temperature and 20 mbar to give 20 g of the title compound in analytically pure form.

Version 2: Cleavage of Phthalimide with KOH

Solid KOH (20 g) is added to a suspension of 4-(3-pyridyl)1H-imidazol-1-butanamide phthalimide (20 g) in water (100 mL). The mixture is refluxed until completion of the reaction is detected by HPLC analysis. After cooling to 25° C. dichloromethane (320 mL) is added and the phases are separated and the aqueous layer is washed with dichloromethane (160 mL). The combined organic phases are extracted with 4N aqueous HCl (58 mL). The water extract is slowly added to 2-propanol (650 mL) at 45° C. The resulting suspension is aged for 2 h at ambient temperature and for another hour at 0° C. The crystals are collected by filtration, washed with 2-propanol and dried at ambient temperature and 20 mbar to give 17.2 g of the title compound in analytically pure form.

¹³C-NMR (DMSO-d₆, 30 mg/0.7 ml, 23° C.): 144.42, 141.46, 139.67, 138.25, 129.67, 127.47, 127.40, 121.68, 48.81, 38.60, 27.01, 24.19 ppm.

XRD spectrum of 4-[4-(3-pyridyl)imidazol-1-yl]butylamine*3HCl*2H₂O (2-θ values): 6.5; 13.0; 19.5; 20.0; 20.8; 22.4; 23.6; 25.5; 26.1; 27.0; 27.9; 28.5; 29.4; 30.2 (FIG. 6).

### Example 5

### Crystallization of 2′-O-Acetyl-3-de[(2,6-dideoxy-3-C-methyl-3-O-methyl-α-L-ribohexopyranosyl)oxy]-11,12-dideoxy-6-O-methyl-3-oxo-12,11-[oxycarbonyl[[4-[4-(3-pyridinyl)-1H-imidazol-1-yl]butyl]imino]]erythromycin (compound 22a)

Crude 22a (8.0 g, obtained according to Ex. 3) was dissolved in isopropyl acetate (15 mL) at 40° C. The solution was treated with 40 mL diisopropyl ether, seeded and slowly cooled. The crystals were collected by filtration, washed and dried to give the title compound in analytically pure form.

¹³C-NMR (CDCl₃): 216.67, 203.92, 170.20, 157.69, 148.00, 146.85, 139.51, 138.17, 132.36, 130.68, 123.88, 115.90, 101.98, 82.58, 78.90, 78.62, 77.99, 71.93, 69.59, 63.79, 60.72, 51.63, 50.11, 47.49, 47.24, 45.28, 42.98, 41.03, 39.50, 39.38, 30.75, 28.98, 24.70, 22.70, 21.82, 21.39, 20.17, 18.78, 16.01, 15.14, 14.47, 14.31, 10.91 ppm.

mp: 191° C.

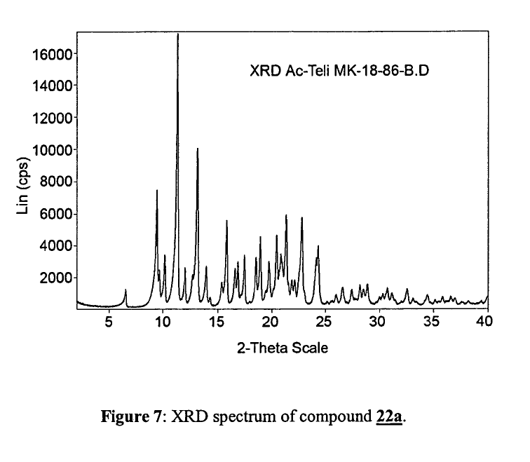

XRD spectrum of compound 22a (2-θ values): 6.6; 9.4; 9.7; 10.2; 11.3; 12.1; 13.1; 14.0; 15.4; 15.8; 16.6; 16.9; 17.5; 18.6; 19.0; 19.7; 20.5; 20.8; 21.3; 21.9; 22.1; 22.8; 24.3; 26.6; 27.4; 28.2; 28.9; 30.7 (FIG. 7).

1-16. (canceled)

17. A process for the preparation of telithromycin comprising:
a) oxidizing a compound of formula 19 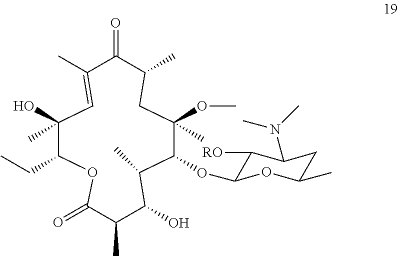
where R is a hydroxyl protecting group, and isolating a compound of formula 20 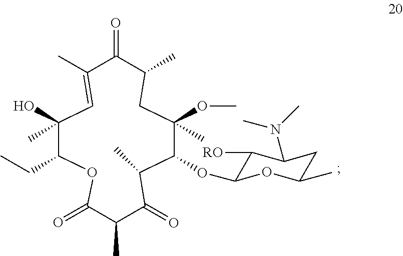
b) reacting the compound of formula 20 with carbonyldiimidazole in the presence of a base to obtain a compound of formula 21 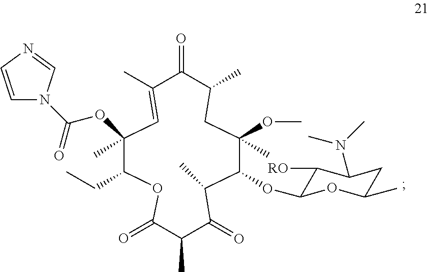
c) condensing the compound of formula 21 with a compound of formula 18 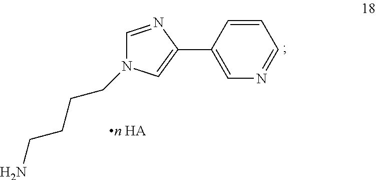
where n is number from 1 to 4 and HA represents an inorganic or organic acid, in the presence of a base to form a compound of formula 22 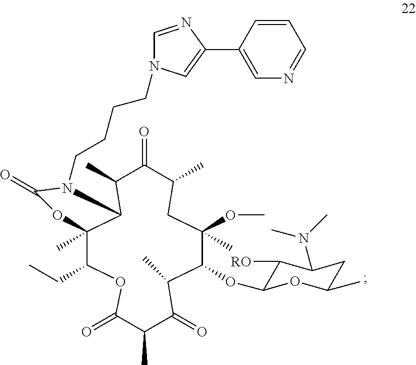
d) deprotecting the compound of formula 22 by removing the hydroxyl protecting group R and isolating telithromycin of formula 1 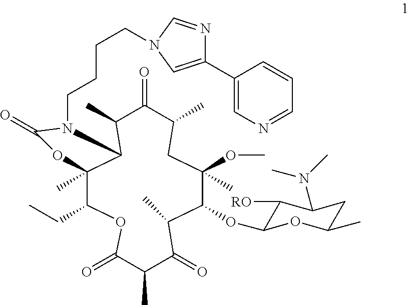
as a single isomer; and
e) crystallizing thus obtained telithromycin of formula 1 to provide telithromycin in high purity.

18. The process according to claim 17, wherein the compound of formula 19 is prepared from clarithromycin in crystalline form.

19. The process according to claim 17, wherein the compound of formula 20 is isolated in crystalline form.

20. The process according to claim 17, further comprising preparing the compound of formula 18 by cleaving phthalimide of formula 17 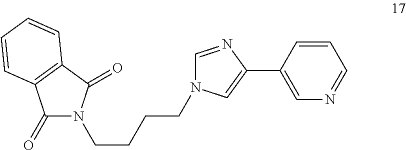
by any means prior to treating the resulting amine with an aqueous acid to form the compound of formula 18.

21. The process according to claim 17, further comprising preparing the compound of formula 18 by directly treating phthalimide of formula 17 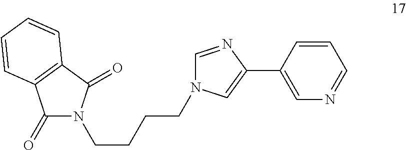
with an aqueous acid to form the compound of formula 18.

22. The process according to claim 17, further comprising preparing the compound of formula 18 by cleaving phthalimide of formula 17 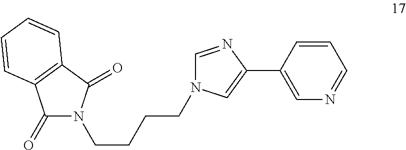
with a base prior to treating the resulting amine with an aqueous acid to form the compound of formula 18.

23. The process according to claim 22, wherein the aqueous acid is aqueous HCl.

24. The process according to claim 17, wherein the compound of formula 22 is isolated in crystalline form.

25. The process according to claim 17, wherein the condensation of step c is conducted in the presence of a base.

26. The process according to claim 25, wherein the base comprises an organic base.

27. The process according to claim 25, wherein the base is DBU or TMG.

28. Crystalline compound of formula 19 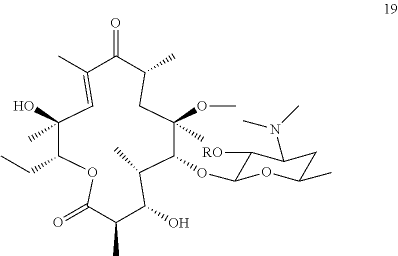
where R is acetyl, having an XRPD pattern with peaks at 11.7, 15.6, 18.5, 19.8, 20.4, ±0.2° 2-θ values.

29. Crystalline compound of formula 20 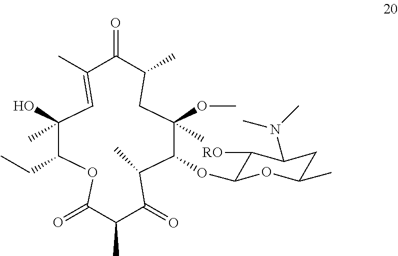
where R is acetyl, having a XRPD pattern with peaks at 11.8, 15.5, 18.5, 19.8, 20.5, ±0.2° 2-θ values.

30. A compound of formula 18 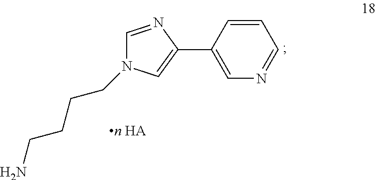
wherein n represents a number from 1 to 4, and HA represents an inorganic or organic acid.

31. A compound of formula 18 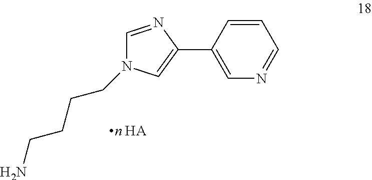
which is 4-[4-(3-pyridyl)imidazol-l-yl]butylamine×3HCl×2H₂O.

32. Crystalline 4-[4-(3-pyridyl)imidazol-l-yl]butylamine×3HCl×2H₂O, having a XRPD pattern with peaks at 19.5, 25.5, 26.1, 27.9, 28.5±0.2° 2-θ values.

33. Crystalline compound of formula 22 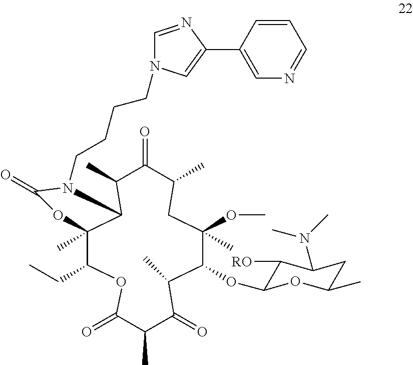
where N is AC, having a XRPD pattern with peaks at 9.4, 11.3, 13.1, 21.3, 22.8±0.2° 2-θ values.

34. A method of synthesizing telithromycin comprising using a compound of formula 18 [chemical structure]
